SEQUENCE LISTING (2 sequences)

SEQ ID NO: 1 - DNA, 19 bp, Artificial Sequence
  Description of Artificial Sequence Synthetic primer
cgctcgttgcgggacttaa

SEQ ID NO: 2 - DNA, 21 bp, Artificial Sequence
  Description of Artificial Sequence Synthetic primer
gagagtttgatcntggctcag

(19) United States
(12) Patent Application Publication
(10) Pub. No.: US 2011/0159488 A1
(43) Pub. Date: Jun. 30, 2011

(54) Primers for Amplification and Sequencing of Eubacterial 16S rDNA for Identification

(75) Inventor: Stefan Emler, Zurich (CH)

(73) Assignee: SmartGene GmbH, Zug (CH)

(21) Appl. No.: 11/887,695

(22) PCT Filed: Apr. 1, 2005
(86) PCT No.: PCT/CH2005/000190

§ 371 Date: Oct. 1, 2007

(51) Int. Cl. C12Q 1/68; C12P 19/34; C07K 14/00; C07H 21/04

(52) U.S. Cl. 435/6.11; 435/91.2; 530/395; 536/23.1

(57) ABSTRACT

The invention relates to oligonucleotides for the qualitative and/or quantitative amplification and/or the sequencing of 16S rDNA-genes, as well as of fragments thereof and RNA derived thereof. It relates to their use as primers in amplification reactions and in sequencing, in particular in combination for the identification of the genus/species/strain of the bacterial sample or clinical isolate.

### TECHNICAL FIELD

The invention relates to oligonucleotides and their use for the qualitative and/or quantitative amplification and/or the sequencing of 16 S rDNA-genes, as well as of fragments thereof and RNA derived thereof. It in particular relates to the identification of bacteria.

### BACKGROUND OF THE INVENTION

Bacterial contamination of food, water, or soil can result in serious illness in humans and animals. Detection and identification of pathogenic organisms are important for containment of potential epidemics, for elimination of natural host reservoirs, for prevention of further contamination, and for appropriate subsequent treatment should exposure occur. Rapid detection and identification of the sources of contamination provides the information to properly eliminate and prevent the spread of bacteria so as to ensure the quality and safety of food and water resources, and the prevention of epidemics.

The same applies if a human or animal has been infected by bacteria, also here the rapid detection and specific identification of the bacterial species or strain from clinical specimens is key to subsequent treatment.

For the identification of bacteria in various media classically cultivation of the bacteria is used for amplification and subsequent depiction/identification. However, cultivation of the bacteria is time-consuming and in particular, in many cases neither the species, and even less the strain can be safely and unambiguously determined.

The polymerase chain reaction (PCR) is an in-vitro method of amplifying DNA sequences. Target DNA from a bacterial source of interest may be amplified and detected in minute quantities. The target DNA to be amplified must be flanked by a known sequence of several nucleotides; short pieces of DNA are synthesized with sequences identical to the known sequences; these are referred to as oligonucleotide primers or simply primers. The PCR process usually requires denaturing the target DNA strand into two separate strands by heating it to 90-98° C. A predetermined primer is then annealed to each of the separate strands at the flanking positions under hybridisation conditions. A heat-resistant enzyme, referred to as Taq polymerase, synthesizes a strand of DNA complementary to the existing DNA strands to form two complete double DNA copies of the original starting target DNA. By repeating this process once, four double-stranded chains are formed. Every time the process is repeated, the amount of PCR product is exponentially increased.

The amplified PCR product can subsequently be used, i.e. subjected to specific marker systems, for example specific sequences which hybridise with genes of specific bacterial strains, in order to identify the strain.

### SUMMARY OF THE INVENTION

The objective problem underlying the present invention is therefore to provide improved systems of oligonucleotides for the identification of the bacterial genera/species/strains present in particular in a clinical specimen or in bacterial cultures, as well as methods for using these oligonucleotides for the identification. In particular the invention relates to oligonucleotides for the qualitative and/or quantitative amplification and/or the sequencing of 16S rDNA-genes, as well as of fragments thereof and RNA derived thereof.

The present invention solves the above problem in that primers are being used which are only hybridizing with regions of the 16S rDNA-gene which are highly conserved. The primers are aiming at binding to these highly conserved regions and when using the PCR amplification at generating amplicons which comprise strain specific regions between the conserved regions to which the primers are binding such that for example using sequencing of the full amplicons these strain specific regions can be analysed and correspondingly the bacterial system identified unambiguously.

So the primers aiming at highly conserved regions of the 16S rDNA or 16S rRNA allow broadband PCR amplification, i.e. amplification which is substantially independent of the genera/species/strains of the bacterial cultures to be identified. On the other hand these highly conserved regions of the 16S rDNA or 16S rRNA enclose highly strain-specific regions, which will be reflected in the generated amplicons. Sequence analysis of the amplicons correspondingly allows identification of the genera/species/strains of the bacterial cultures based on the sequence of these highly strain-specific regions which are present in the fragment. As a matter of fact, the proposed primers have been tested with more than 800 clinical isolates of more than 80 genera and more than 190 different species, and are surprisingly working for this vast group of bacterial systems proving that their functioning is substantially independent from the bacterial genus, allowing broadband detection/amplification etc. The primers may be used for amplification, sequencing, marking, as probes etc.

The proposed identification of bacteria by 16S rDNA sequence analysis allows identification of cultures within 24 h, regardless of growth or metabolism and can identify fastidious and novel microorganisms.

The object of the present invention are therefore oligonucleotides selected from an oligonucleotide comprising the sequence called BR16SR (SEQ ID No. 1 as given in the attached sequence listing):

CGCTCGTTGC GGGACTTAA (5′-3′; l9 mer; reverse)

and/or an oligonucleotide comprising the sequence B162 (SEQ ID No. 2 as given in the attached sequence listing):

GAGAGTTTGA TCNTGGCTCA G (5′-3′; 21 mer, forward)

wherein N stands for the placeholder Inosine.

As well, their complementary oligonucleotides, reverse oligonucleotides, reverse complementary oligonucleotides, RNA derived thereof, which are selectively hybridizing to these specific, highly conserved regions of the 16S rDNA-genes, and also fragments thereof and RNA derived thereof, are the object of the invention.

Furthermore, functionally equivalent variants shall also be included, namely derivatives thereof, in which 1, 2, 3 or at most 4 nucleotides are replaced by another nucleotide (or by a placeholder), added or deleted without substantially amending the selective hybridization to these specific regions of the 16S rDNA-genes, as well as to fragments thereof and RNA derived thereof. Preferred are the above sequences which are completely unmodified, but e.g. also up to two or three nucleotides may be added terminally at each end without shifting Tₘ too far out of the practical ranges and, if the additions are carefully chosen, without amending the specificity substantially. Preferred are oligonucleotides in which not more than 1 nucleotide is replaced in SEQ ID No. 1 and/or SEQ ID No. 2 by another nucleotide (or by a placeholder), and/or not more than 1 nucleotide (or a placeholder) is added or deleted. In this case, these amendments to the sequence shall also only be included if they do not substantially amend the selective hybridization to these specific, highly conserved regions of the 16S rDNA-genes, as well as to fragments thereof and RNA derived thereof. Also possible and functionally substantially identical is a wobble at the position of N=Inosine with A and C. Of course also such a wobble is included.

Preferably, the selective hybridisation takes place under stringent conditions only, wherein for example under PCR using recombinant DNA polymerase from Thermus aquaticus the stringent conditions are defined as: 50-60° C., at 500 nM MgCl₂.

According to a preferred embodiment, the oligonucleotides consist of the above-mentioned oligonucleotides SEQ ID No. 1 and/or SEQ ID No. 2 as well as of their complementary oligonucleotides, reverse oligonucleotides, reverse complementary oligonucleotides, RNA derived thereof, which are selectively hybridizing, preferably under stringent conditions, to specific regions of the 16S rDNA-genes, as well as to fragments thereof and RNA derived thereof.

According to the present invention, surprisingly two highly conserved regions could be found which embrace a strain specific strand of approximately 1000-1200 bases, and which are located at the positions (−3) to 18 and 1098 to 1080 as defined in E. Coli X80724 (using a different reference system, the positions may change). Correspondingly, according to the present invention, preferably the oligonucleotide comprising the sequence SEQ ID No. 1 under stringent conditions specifically hybridises with the specific region from 1098 to 1080 as defined in E. Coli X80724, and the oligonucleotide comprising the sequence SEQ ID No. 2 under stringent conditions specifically hybridises with the specific region from (−3) to 18 as defined in E. Coli X80724. Since the positioning depends on the reference system, also shiftings by no more than 10, preferentially shiftings by not more than 4 nucleotides in respect to these specific regions should be taken account of.

The oligonucleotides may comprise at least one marker and/or they may be attached to a matrix for a specific purposes.

For amplification purposes using PCR, the above-mentioned oligonucleotides according to sequences SEQ ID No. 1 and SEQ ID No. 2 are preferably used as a pair in order to generate the desired fragment which is bordered by the two primers. Preferentially, such a mixture of the two primers comprises these oligonucleotides (and/or the complementary oligonucleotides) in equal amounts.

Furthermore, the present invention also relates to a method for the specific amplification and/or the sequencing of 16S rDNA-genes, as well as of fragments thereof and RNA derived thereof, wherein as primer at least one oligonucleotide as given above or a mixture as given above are used.

In case of amplification of the 16S rDNA-gene, or the fragments thereof or the RNA derived thereof the oligonucleotides are brought into contact with a mixture of the pair of the distinct oligonucleotides SEQ ID No. 1 and SEQ ID No. 2 (and/or the complementary oligonucleotides) acting as reverse and forward primer, respectively, and are subjected to a polymerase chain reaction leading to specific marker fragments with in the range of 100-2000, preferentially in the range of 800-1200 nucleotides. Contact between the 16S rDNA-gene, or the fragments thereof or the RNA derived thereof, with a mixture of the pair of the distinct oligonucleotides SEQ ID No. 1 and SEQ ID No. 2 (and/or the complementary oligonucleotides) is preferably established under stringent conditions, which in case of PCR using recombinant DNA polymerase from Thermus aquaticus the stringent conditions are defined as: 55° C., 500 nM MgCl₂.

Typically, in a first step the 16S rDNA-gene, or the fragments thereof or the RNA derived thereof is heat-denaturated in order to obtain single-stranded chains, subsequently these are brought into contact with a mixture of the pair of the distinct oligonucleotides SEQ ID No. 1 and SEQ ID No. 2 (and/or the complementary oligonucleotides), and then extended by means of a DNA polymerase, and wherein the heat-denaturation and the extension cycles are repeated in order to obtain a detectable amount of product.

Furthermore, the present invention relates to the use of the above-mentioned oligonucleotides for sequencing purposes. Correspondingly, a method is proposed for the sequencing using the chain termination method (or Sanger method), in which a sample is sequenced in both directions using either the reverse, SEQ ID No. 1, or the forward, SEQ ID No. 2 (and/or the complementary oligonucleotides), as a primer. Also here, preferably for the amplification steps in the sequencing reaction stringent conditions are used, which e.g. means that under PCR using recombinant DNA polymerase from Thermus aquaticus the stringent conditions are defined as: 52° C., 500 nM MgCl₂.

In addition to that, presently a method is proposed for the identification of bacterial species based on their 16S rDNA-gene. This method uses the following steps a) sample material of a bacterial colony is isolated and the genetic material is extracted or at least made accessible to amplification; b) the thus derived material is subjected to amplification in accordance with the method as described above; c) the double-stranded DNA amplification products are purified; d) the purified product is subjected to cycle-sequencing using a method as described above, preferably with the chain termination principle (for the sequencing however in step d), also completely different sequencing methods may be used); e) the sequenced sample is purified; f) the purified and sequenced sample is subjected to sequence electrophoresis and signal recording. Preferably, for the initial amplification in step b) as well as for the sequencing in step d) the same set of primers (and/or the complementary oligonucleotides) as given above is used, i.e. for the initial amplification the pair of primers is concomitantly used for generating the fragment of preferably approximately 1030 bp, and in the two sequencing reactions (from both sides) either of the two primers is used.

For the identification of the bacterial genus/species/strain present in the bacterial colony the raw sequence data as obtained in step f) are preferably automatically aligned, noise sequences are optionally stripped, a consensus sequence is created by comparing the measured data with sequence data from references from a database of known bacterial genera/species/strains, all this using computer implemented methods.

Further embodiments of the present invention are outlined in the dependent claims.

### SHORT DESCRIPTION OF THE FIGURES

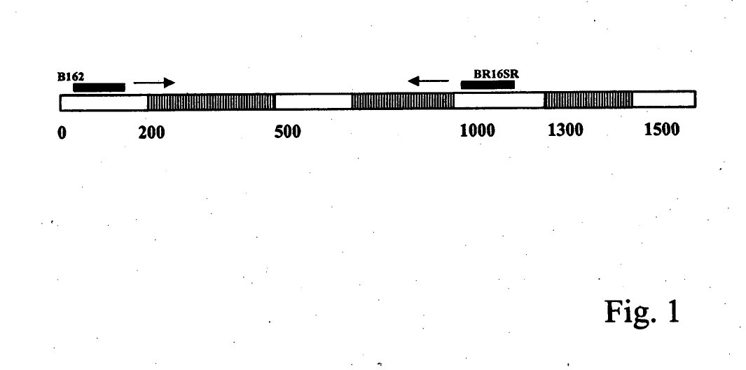

In the accompanying FIG. 1, the fragment of 16S rDNA/rRNA as chosen for analysis is displayed schematically with variable and conserved regions as well as the positions of the primers are indicated.

### DETAILED DESCRIPTION OF THE PREFERRED EMBODIMENTS

The gene for the 16S subunit of the bacterial ribosome is highly conserved over several stretches in all eubacteria. Using the specific primers according to the present invention, which are named B162 (SEQ ID No. 2) and BR16SR (SEQ ID No. 1) to conserved regions in all known bacteria, a broad-range PCR allows amplification of a >1000 bp fragment of the 16S rDNA starting with crude DNA-preparations out of any suitable bacterial culture.

In this respect see FIG. 1 which displays this stretch of the gene, and in which hatched regions indicate possible strain-specific regions (their position may vary) and in which white areas show the conserved parts.

For a secure identification of the bacterial isolate amplified, the same primers are preferentially as used for amplification of the genetic material are applied to direct sequencing of the amplified product. e.g. using the Sanger method or equivalent methods which rely on the use of a primer. Any commercially available direct-sequencing technology or DNA sequencer is suitable for this purpose.

Since the positions of the so-called “species-specific variable regions” (hatched in FIG. 1) vary between the different bacterial genus, it is important that the amplification primers span a region large enough to contain at least one or two variable regions within the fragment with sufficient differentiation with regard to a to-be-identified bacterial isolate. The fragment of about 1030 bp presently opted for demonstrably contains this variability for the differentiation of almost all clinically relevant bacterial species.

Technically, a fragment of 1000 bp can be sequenced from both sides with only two sequencing reactions (using the amplification primers) and therefore saves costs, time and material while preserving high analytical accuracy; with ongoing progress in sequencing technology it is conceivable that only one sequencing reaction may be applied in the future.

A vast range of different genera (more than 80) and corresponding species (more than 190) has been tested on more than 800 clinical isolates to be identifiable with the proposed primers. A list is given further below.

The primers B162 (SEQ ID No. 2) and BR16SR (SEQ ID No. 1) are both designed to hybridize specifically to conserved parts of the bacterial 16S rRNA gene (16S rDNA) and to produce a fragment of about 1030 bp, including variable regions independently of their position within this fragment. In order to avoid misalignment, one primer B162 (SEQ ID No. 2) includes a non-specific placeholder N (Inosin) instead of a A, C, G or T.

In Table 1 the sequences of the two primers are given as well as their positioning within the 16S rDNA gene of Escherichia coli NCBI X80724, and their annealing temperatures as calculated using the Wallace formula (Tₘ=2(A+T)+4(G+C)), wherein Inosine is counted as T/A.

TABLE 1

| Oligonu- | | | | |
|---|---|---|---|---|
| cleotide | SEQ ID | Sequence¹ | Position² | Tₘ |
| BR16SR | No. 1 | cgctcgttgc | 1098-1080 | 60° |
| | | gggacttaa | | |
| B162 | No. 2 | gagagtttga | (−3) to 18 | 60° |
| | | tcntggctca g | | |

¹n = Inosine
²Position in the 16S rDNA gene of Escherichia coli NCBI X80724.

For a secure identification of the sequencing data, only material from one strain/colony should be used; mixed cultures and contaminated primary material may result in “unreadable” overlay sequences and should be avoided. See below for details on the preparation of different samples. If other technologies than direct sequencing are applied to differentiate the variable species-specific regions (e.g. micro-array, hybridization, cloning and sequencing . . . ), this restriction to single (pure) colonies does not apply.

In the following, the specific procedure for amplification and subsequent sequencing for identification of eubacterial 16S rDNA is given, however, this specific example should not be used to limit the scope of the invention as claimed in the appended claims. In particular variants thereof which are within the scope of the general knowledge and practice of the person skilled in the art are explicitly included.

### 1. Testmaterial/Software

Kit: Big-Dye Terminator Cyle Sequencing (Applied Biosystems Art.-Nr. 4303152)
Kit: QIAamp DNA mini kit (Qiagen Art.-Nr. 51306)
Kit: QIAquick PCR purification kit (Qiagen Art.-Nr. 28106)
Kit: DyeEx spin kit (Qiagen Art.-Nr. 63106)
Heating-block 95-96° C.
Ultrasonic unit (e.g. Abbott “LCX Lysor”)
Benchtop centrifuge (e.g. Eppendorf 5415D)
Thermocycler (e.g. PE 9600)
Equipment for gel-electrophoresis, including gel-trays with casting-facility, power supply, staining/destaining trays, transilluminator, Polaroid-camera and specific reagents
Speedvac (e.g. Eppendorf Concentrator 5301)
Sequencing-device (e.g. ABI/PE310) including peripheral equipment and reagents (long capillary, tubes, caps, polymer=POP-6, seq. buffer 10×, template suppression reagent=TSR)
Tubes 1.5 ml (Eppendorf and Sarstedt) and 0.2 ml (Sarstedt multiply pro) with tubeholders
H₂O HPLC grade, sterile; TE pH 8.0/7.5 (10 mM Tris·Cl, 1 mM EDTA); 0.9% NaCl sterile
Glass beads, acid washed (Sigma G-4649),
Ampli-Taq LD (Applied Biosystems Art.-Nr. N808-0107)
Primers for eubacterial 16s rRNA broad-range PCR, specifically B162 (SEQ ID No. 2) and BR16SR (SEQ ID No. 1).
Software: 310 Collection; Sequencing Analysis; MT Navigator PPC, IDNS™ by SmartGene, with account
   2. Sample material
1 loop of a bacterial colony from a culture dish, suspended in TE pH 8.0 or out of liquid culture; avoid agar.
Primary material can be used in certain cases (i.e. when collected under sterile conditions, as for CSF or joint-punctuations). Contaminated starting material such as sputum results in multiple, unreadable overlaid signal peaks.

### 3. Preparation of Reagents:

Use sterile filtertips on all clinical samples/cultures/DNA-preparations in order to avoid contamination. Label all reagent-tubes. All clinical, material has to be considered as potentially infectious and should be handled in laminar flow benches only unless inactivated at 95° C. for at least 15 min.
   3.1. General preparations'
Extraction: preheat heating-block to 96° C. Amplification: pre-heat the thermocycler, pour a 2% agarose-gel and let it cool down for at least 2 h. Cycle-sequencing: pre-heat the thermocycler. Sequencing: switch on speedvac (30° C.), equilibrate Template Suppression Reagent (TSR) and 1× Seq.-Puffer to RT, preheat heating-block to 96° C., switch on DNA-Sequencer and computer.

### 3.2. Sample Preparation

Suitable bacterial colonies on solid medium are transferred without any culture medium (possible inhibition of PCR) from the culture to a 1.5 ml Sarstedt screw-cap tube, previously filled with 300 ml TE, mix gently. Bacterial suspensions can be stored at 4° C. for a maximum of 5 d.
Liquid-cultures: transfer approx. 1 ml of bacterial suspension to a 1.5 ml Sarstedt tube and centrifuge for 5 min. at 5000 rpm. Discard the supernatant in the laminar-flow bench using filter-tips and resuspend the pellet in 500 ml 0.9% NaCl. Spin down, discard supernatant and resuspend the pellet in 300 ml TE, then follow standard-procedure.
Primary specimens, like whole blood, CSF (CerebroSpinal Fluid), liquid from pleural-punctures may be used only if it can be assumed that just one predominant germ is present and contamination by flora can be excluded. Material should be treated similarly to the liquid-culture procedure.

### 4. Extraction

Use the QIAamp DNA mini kit for the extraction of clinical specimens or for culture suspensions containing inhibitory substances such as agar etc.
1. Gently mix the bacterial suspension in the closed screw-cap Sarsted tubes and incubate them for 15 min. at 96° C. in order to inactivate the bacteria. Cool down for 5 min. at RT, mix and quick-spin, in order to avoid lid contamination.
2. Add approx. 100 ml glass-beads (Sigma) by using a 1000 ml pipette with filter tip (fixed to 500 ml, “dry” pipetting). Close caps and vortex briefly.
3. Place all sample tubes symmetrically on the dry sonicator Lysor-plate, equilibrate with mock-tubes if necessary. Sonicate for approx. 15 min., when finished, remove tubes; vortex and spin down the glass-beads (5 min. at 13,000 rpm).
4. Immediately transfer the supernatants into new 1.5 ml Eppendorf tubes in order to avoid adsorption of DNA to glass-beads.
5. Qiagen-extraction: samples out of liquid-cultures and potentially inhibited samples should be further purified using silica-columns. For each sample use 200 ml of supernatant post sonication and follow the Qiagen protocol (QIAamp DNA mini kit).

### 5. Amplification

Standard PCR-master-mix can be prepared previously, including the LD-AmpliTaq.
1. Instruction for master-mix: See Table 2 for a typical pipetting setup of single components useful for one test.

TABLE 2
Master-mix-setup for broad-range PCR using standard primers

| | conc. | | volume | | final | |
|---|---|---|---|---|---|---|
| components | stock sol. | | (one test) | | conc. | |
| H₂O HPLC-grade | — | | 58.77 | μl | — | |
| reaction buffer (PE) | 10.00 | x | 10.00 | μl | 1.00 | x |
| MgCl₂ (1) | 150.00 | mM | 0.33 | μl | 500.00 | nM |
| dNTP | 2.00 | mM | 25.00 | μl | 500.00 | μM |
| primer B162 (2) | 50 | μM | 0.20 | μl | 100.00 | nM |
| primer BR16SR (2) | 50 | μM | 0.20 | μl | 100.00 | nM |
| AmpliTaq LD (PE) | 5.00 | U/μl | 0.50 | μl | 2.50 | U/rx |
| Total vol. without | | | 95.00 | μl | | |
| template | | | | | | |

(1) including. 1.5 mM already contained in the reaction buffer,
(2) concentration per ml TE pH 7.5

2. The corresponding broad-range PCR for mycobacteria (master-mix EU2) with B162 and BR16SR is thawed, mixed briefly, centrifuged and pipetted in aliquots of 95 ml into labelled 0.2 ml PCR single-tubes (Sarstedt Multiply pro). Include one negative, one positive control to each amplification; potentially inhibited samples or primary clinical specimens may require a spiked inhibition control or a dilution 1:5.
3. For the negative control, add 5 ml of H₂O(HPLC-grade) to the master-mix.
4. For the positive amplification-control or for inhibition control, add 5 μl of previously successfully amplified DNA to the master-mix.
5. Add 5 ml of each sample DNA-suspension to the master-mix, close caps immediately after pipetting in order to avoid cross contamination.
6. Amplification on a thermocycler (ex. PE 9600): 3′ 95° C., 38×[30″ 95° C., 30″ 55° C., 45″ 72° C.], 5′ 72° C., 99 h 5° C.
7. After PCR, caps of PCR tubes are opened one by one and 10 ml of each amplification product is analyzed on a 2% agarose-gel, stained with ethidium-bromid and visualized on a transilluminator. Only samples with clear bands of the proper size should be used for further processing.
   6. Purification after amplification
Use the QIAquick PCR purification kit for direct purification of double-stranded (ds) DNA PCR amplification products. This procedure is mainly the manufacturer's recommendation for purification of PCR products for sequencing purposes.
1. Spin down all PCR-amplicons in 0.2 ml single tubes (cf 10″ at ≧10′000×g).
2. Pipet 500 ml (5 Vol.) of PB buffer (Qiagen) for each sample into a clean, labelled 1.5 ml reaction tube, then add the PCR-amplicons (approx. 90 ml) using filtertips. Mix briefly on a vortex. For 50 ml PCR volume, adjust PB buffer accordingly.
3. Label the QIAquick columns and put them in the collection tubes provided with the kit
4. Quick-spin all samples; transfer liquid on the corresponding QIAquick spin column (use filter-tips); amplicons will bind to the silica-matrix.
5. Spin for 60 sec. at ≧10,000×g (approx. 13,000 rpm on a Eppendorf centrifuge 5415D).
6. Transfer the QIAquick column to a fresh collection tube, discard the eluate.
7. Wash the QIAquick columns by adding 750 ml PE buffer (Qiagen, must be diluted in advance with 99% ethanol HPLC according to instructions) and spin for 60 sec. at ≧10,000×g
8. Again, transfer the QIAquick column to a fresh collection tube and discard the eluate. Spin for 60 sec. at >10,000×g. This additional-centrifugation step is needed for complete removal of any residual ethanol.
9. Transfer the QIAquick column to a fresh, labelled 1.5 ml reaction tube and discard the eluate.
10. To elute the amplicon, pipet 50 ml EB Puffer (Qiagen, 10 mM Tris, pH 8.5) directly to the center of the QIAquick membrane. In order to increase the concentration of amplicons, use only 30 ml EB and incubate at RT for 1 min. prior to centrifugation for 60 sec. at ≧10,000×g. Remove column and store purified PCR products up to 7d at +4° C., or at −20° C. for longer periods.

### 7. Cycle-Sequencing

The AB DNA Big Dye Terminator. Sequencing Kit can be used to sequence and end-label purified broad-range PCR products with BigDye-fluorescence markers. Same primers, i.e. B162 and BR16SR, as for PCR are used in order to sequence the entire amplicon, to use one primer per reaction.
1. Each sample is sequenced in both directions using forward (B162) and reverse primers (BR16SR). As positive control for the sequencing reaction, the plasmid provided with the sequencing kit can be used, together with the kit-primer.
2. The following components are pipetted into 0.2 ml PCR tubes (Table 3): H₂O HPLC grade, ad 20 ml, 4 ml Cycle-Seq master-mix, 1 ml primer B162 or BR16SR (10 μM) and 1-10 ml purified broad-range PCR product (depending on the signal on the gel). Double volume of the mastermix to 8 ml when sequencing samples with faint signals on gel.

TABLE 3
Pipetting-scheme for the setup of a BigDye Terminator Cycle
sequencing reaction. “Pos. Seq” relies on the position of the sample on the
48-sequencing-tray of the PE310.

| No. | Pos. | Gel: | dir. | Vol. | | Vol. | MMx | H₂O | total |
|---|---|---|---|---|---|---|---|---|---|
| tub | Seq. | slot | (f/r) | [μl] | primer | [μl] | [μl] | [μl] | [μl] |
| 2 | A3 | xx | f | 1 | B162 | 1 | 4 | 14 | 20 |
| 3 | A5 | xx | r | 1 | BR16sr | 1 | 4 | 14 | 20 |
| 4 | A7 | yy | f | 5 | B162 | 1 | 8 | 6 | 20 |

3. Amplification on a thermocycler: 1′ 96° C., 25×[10″ 96° C., 5″ 52° C., 4′ 60° C.], 99 h 4° C. The Cycle-sequencing amplification products may be stored up to 5d at 4° C., protected from light.
   8. Purification of the sequenced sample
The QIAGEN DyeEx Spin Kit is used to remove unincorporated Big-dye ddNTP from the sequencing reaction.
1. Carefully spin down sample tubes (60 sec. at ≧10,000×g).
2. For each sample to be purified label a gel-column (red cap), invert briefly, flip of lower closure and loosen cap a quarter of a turn.
3. Centrifuge the columns for 3 min. at 750×g (3000 rpm on a Eppendorf centrifuge 5415D) and transfer them into 1.5 ml reaction tubes.
4. Carefully pipet the sequencing-reaction-mixtures (10-20 ml) on the corresponding columns, avoiding to touch neither the column-walls nor the gel-slurry. Samples with a volume lower than 10 ml have should be expanded to 20 ml with H₂O HPLC grade.
5. Centrifuge the columns (cf 3 min. at 750×g), the eluate contains the sequencing-sample. Discard the column.
6. Dry all samples for 30 min. at 30° C. in a speedvac (caps toward center). Dried samples are stable indefinitely at 4° C., light-protected.
   9. Sequence electrophoresis and signal recording
A capillary-sequencer such as the ABI Prism310 Genetic Analyzer can be used.
1. Rinse capillary first, use yellow tape to fix it approx. 0.5 mm below the anode, then switch on sequencer and computer. The apparatus initializes and homes automatically syringe and autosampler. Calibration of the capillary is required only after replacement of the anode or the capillary. Set capillary to position 3 (H₂O, “Autosampler To Position”, then “Autosampler Up” approx. 500 steps until end dips in completely).
2. The lyophilized samples from the cycle sequencing reaction are resuspended in 25 ml TSR (Template-Suspension Reagent), mixed well and briefly centrifuged (quick-spin).
3. Denature all samples for 2 min. at 95° C. and chill down immediately on a cooler. Mix well and quick-spin. The samples are then transferred into labelled sequencing tubes using filter tips, closed with rubber stoppers and placed onto the 48-rack at the same positions as indicated in the injection list. The rack is fixed onto the autosampler and both doors are closed after a second push on “TRAY”.
4. Sample Sheet (File/New “Sequence Smpl Sheet 48 Tube”): position A1 is always CCD (prerun testing of the photo-detectors), then all samples according to the injection list. Choose the Dye Set/Primer “DT POP6 {BD Set-any Primer}” and Matrix “Seq Matrix E”.
5. Injection List: Length to Detector=50 cm (long capillary). Controls: CCD-Test (set module to “CCD pre-run”) always at the beginning, pGEM—if needed—at the end of a sequencing run.
6. Start the sequencing. CCD-test should result in signal curves below 2000, if the values are too high capillary and laser-window have to be cleaned carefully using H₂O and 70% EtOH.
7. Additional samples can be inserted during the run if desired.
8. Place the new tubes on the rack of the autosampler and close doors to resume the run automatically.
9. Once the run is finished, remove all tubes from the rack and store at 4° C. until successful analysis of the raw data is finished. Park capillary at position 3 (water) and move it down until submerged. Avoid drying out of the capillary.
   10. Analysis of the sequencing data with the ProofReader-IDNS™
Sequencer manufacturer software can be used for analysis. Preferably the software package ProofReader-1DNS™ as available from the applicant is used as it allows the validation of raw data by comparing them to target-specific references. Moreover, this software package allows:
   Direct upload of raw sequence data (electropherograms) from the sequencer of choice.
   Automated alignments of partial sequences (incl. automated reverse complementation).
   Strip noise sequences at both ends of the sequenced fragment.
   Automated creation of a consensus sequence.
   Display of modified (proof-read) positions, amino acid translation.
   Jump to specific zones of interest (e.g. resistance encoding positions . . . ).
One or more sequences can be imported and by using a procedure called “ProofRead”, they are aligned with a single specific reference or with the most appropriate 16S reference automatically selected in a database. This database contains a subset of full length 16S references. A sequence consensus is obtained by comparing samples to the reference. An overview of the relative position of the samples and the reference can be displayed.
On demand, relevant positions for resistance can be denoted in the reference sequence by a colour code.
Single nucleotide fluorescent signal can be individually adjusted allowing a fine tuning of peak intensities (i.e. useful at the end of the sequence). Mismatches between consensus and reference sequence are clearly identified by highlighted positions. Ambiguities between samples sequences can be displayed. Nucleotides changes may be introduced. In case of correction of wrong nucleotides in the sample sequences, the changes are highlighted. Moreover, to facilitate the validation of numerous samples, the consensus is modifiable. In this case, any consensus modification leads to corresponding changes in the samples.
Sometimes, gaps or insertions are wrongly introduced in the sample sequences by sequencer softwares. Thus, they should be fixed by correcting sample sequences. A warning message appearing before saving will inform the user that such positions are still present.
A “realign function” allows the reassessment of the match-pairing between the samples and the reference. This can be performed after correction and validation steps. Another useful tool allows the retrieval of resistance relevant mutations (if defined), mismatches with reference, ambiguity between contigs and the highlight of special zones defined by the customer.
Garbage sequences may be automatically proposed and denoted by a colour. To trim regions encompassing garbage, select the last position before trimming in the nucleotide sequence, click to select upstream or downstream regions to be trimmed, respectively. More than one region could be selected at once.
Once the sequence is validated, the user should “Save” the sequence in IDNS™. This will automatically store the validated sequence in the database. Nevertheless, the user can still revalidate the samples by using the ProofRead link in the sample sequence window. In this case, the same electropherograms files are automatically reloaded.

The presented experimental scheme shall serve to demonstrate and document in reproducible manner that the proposed sequences indeed fulfil the desired functions. Variations thereof are possible to the person skilled in the art without departing from the invention. The explicitly described protocol shall in any case not be interpreted to limit the scope of the invention as defined in the appended claims.

For verification of the broadband applicability of the proposed primers they have been tested experimentally on a huge number of genera and corresponding species. For all the following systems the primers have been found to work, i.e. to amplify the desired ranges efficiently. For each genus the corresponding species which were tested as well as the number of species are given in brackets. The total number of clinical isolates evaluated is 805 corresponding to ca. 190 species or yet undefined species (sp).

Abiotrophia (adiacens 2, sp 3), Acetobacter(sp 1), Acinetobacter (baumannii 1, haemolyticus 2, lwoffii 3, sp 6), Actinobacillus (actinomycetemcomitans 2, sp 2), Actinomyces (birnadii 1, europae 1, israelii 3, meyeri 1, neuii 2, odontolyticus 1, radingae 1, sp 16, turicensis 1), Aerococcus (urinae 9), Aeromonas(hydrophila 1, sp 6, veronii 1), Alloiococcus (otitis 1), Arcobacter (butzleri 1), Arthrobacter (oxydans 1, sp 1), Atopobium (rimae 1), Aureobacterium (sp 2), Bacillus (cereus 1, flexus 1, licheniformis 1, sp 6); Bifidobacterium (sp 4), Bordetella (sp 1), Brachybacterium (conglomeratum 1), Brevibacillus (sp 1), Brevibacterium (casei 1, sp 2), Brucella (sp1), Burkholderia (sp 3), Campylobacter (fetus 2, jejuni 9, sp 1), Capnocytophaga (canimorsus 5, sp 3), Cellulomonas (sp 1), Chryseobacterium (meningosepticum 1), Clostridium (botulinum 2, novyi 1, paraputrificum 1, septicum 1, sp 5, sporogenes 1, symbiosum 1, tertium 1), Corynebacterium (accolens 1, asperum 5, auris 1, macginleyi 2, otitidis 1, propinquum 1, seminale 3, sp 8, striatum 1, ulcerans 1, urealyticum 2, xerosis 2), Dermobacter (sp 1), Desulfovibrio (sp 1), Eikenella (corrodens 3, sp 1), Enterobacter (aerogenes 3, cloacae 2, sp 8), Enterococcus (avium 5, cecorum 1, durans 2, faecium 2, malodoratus 1, sp 11), Escherichia (coli 33), Fusobacterium (sp 2), Gardnerella (vaginalis 1), Gemella (haemolysans 1, sp 2), Gordona (sp 1), Haemophilus (aphrophilus 3, influenzae 4, paraphrophilus 2, sp 12), Helcococcus (sp Kingella (sp 3), Klebsiella (pneumoniae 3, sp 2), Lactobacillus (acidophilus 1, casei 4, delbrueckii 3, gasseri 2, paracasei 1, pentosus 1, salivarius 1, sp 5, zeae 1), Legionella (micdadei 1), Listeria (ivanovii 1), Methylobacterium (sp 2), Micrococcus (sp 2), Moraxella (catarrhalis 5, nonliquefaciens 7, osloensis 3, phenylpyruvica 1, sp 4), Morganella (morganii 2), Mycobacterium (sp 1, alvei 2, aurum 1, avium 2, branderi 1, doricum 1, fortuitum 4, genavense 4, gilvum 3, gordonae-like 1, hassiacum 2, interjectum 3, kansasii 1, lentiflavum 2, monacense 1, neoaurum 1, paratuberculosis 2, scrofulaceum 1, smegmatis 2, sp 53, szulgai 1, tusciae 3, hominis 1, pneumoniae 1), Neisseiria (cinerea 1, meningitidis 206, sp 1), Nocardia (amycolata 1, asteroides 3, brasiliensis 1, nova 2, sp 5), Ochrobactrum (anthropi 1), Oligella (urethralis 1), Pasteurella (canis 1, multocida 1), Pediococcus (acidilactici 1), Peptostreptococcus (anaerobius 1), Propionibacterium (acnes 3, propionicum 1), Proteus (mirabilis 12, penneri 2), Pseudomonas (aeruginosa 8, diminuta 3, sp 1), Ralstonia (sp 1), Rhodobacter (sp 1), Rhodococcus (sp 1)Ruminococcus (gnavus 1), Sanguibacter (suarezii 1) Sarcina (ventriculi 1), Shigella (boydii 1), Staphylococcus (aureus 6, capitis 1, epidermidis 4, haemolyticus 2, hominis 1, sp 2), Stenotrophomonas (maltophila 4), Streptobacillus (moniliformis 1), Streptococcus (anginosus 1, bovis 1, caprinus 1, dysgalactiae 1, gordonii 1, milleri 1, mitis 36, mitis/pneumoniae 4, mutans 1, parasanguis 2, pneumoniae 32, pyogenes 3, salivarius 3, sp 17), Tsukamurella (sp 1), Turicella (otitidis 2), Ureaplasma (urealyticum 2), Variovorax (sp1).

1. An oligonucleotide or mixture of oligonucleotides for the qualitative and/or quantitative amplification and/or the sequencing of 16S rDNA-genes, wherein said oligonucleotide is selected from an oligonucleotide consisting of or comprising the sequence SEQ ID No. 1 and/or an oligonucleotide consisting of or comprising the sequence SEQ ID No. 2 as well as their complementary oligonucleotides, reverse oligonucleotides, reverse complementary oligonucleotides, RNA derived thereof, which selectively hybridize to specific regions of the 16S rDNA-genes, as well as to fragments thereof and RNA derived thereof, as well as derivatives thereof, in which 1, 2, 3 or 4 nucleotides are replaced by another nucleotide, added terminally, inserted or deleted without substantially amending the selective hybridization to these specific regions of the 16S rDNA-genes, as well as to fragments thereof and RNA derived thereof.

2-20. (canceled)

21. An oligonucleotide according to claim 1, wherein not more than 1 nucleotide is replaced in SEQ ID NO:1 and/or SEQ ID NO:2 by another nucleotide, and/or not more than 1 nucleotide is added terminally, inserted or deleted, all these modifications without substantially amending the selective hybridization to these specific regions of the 16S rDNA-genes, as well as to fragments thereof and RNA derived thereof.

22. An oligonucleotide according to claim 1, wherein only one nucleotide is replaced in SEQ ID NO:2 by another nucleotide, and wherein this replacement is at the position of Inosine and is given by a wobble of C or A.

23. An oligonucleotide according to claim 1, wherein selective hybridisation takes place under stringent conditions only.

24. An oligonucleotide according to claim 1, which is selected from an oligonucleotide consisting of the sequence SEQ ID NO:1 and/or an oligonucleotide consisting of the sequence SEQ ID NO:2 as well as their complementary oligonucleotides, reverse oligonucleotides, reverse complementary oligonucleotides, RNA derived thereof, which selectively hybridize to specific regions of the 16S rDNA-genes, as well as to fragments thereof and RNA derived thereof.

25. An oligonucleotide according to claim 1, wherein the oligonucleotide comprising the sequence SEQ ID NO:1 under stringent conditions specifically hybridises with the specific region from 1098 to 1080 as defined in E. coli X80724, and wherein the oligonucleotide comprising the sequence SEQ ID NO:2 under stringent conditions specifically hybridises with the specific region from (−3) to 18 as defined in E. coli X80724, or wherein each of these oligonucleotides under stringent conditions hybridizes with specific regions shifted by not more than 10, preferentially shifted by not more than 4 nucleotides in respect to these specific regions.

26. An oligonucleotide according to claim 1, which comprises and/or is linked to and/or is attached to at least one functional element selected from: marker, carrier, antibody, capture molecule, bead, in particular magnetic bead.

27. An oligonucleotide according to claim 1, which is attached to a matrix.

28. A composition comprising a mixture of oligonucleotides designated as SEQ ID NOS:1 and 2 for amplification using polymerase chain reaction (PCR), as well as their complementary oligonucleotides, reverse oligonucleotides, reverse complementary oligonucleotides, RNA derived thereof, which selectively hybridize to specific regions of the 16S rDNA-genes, as well as to fragments thereof and RNA derived thereof, as well as derivatives thereof, in which 1, 2, 3 or 4 nucleotides are replaced by another nucleotide, added terminally, inserted or deleted without substantially amending the selective hybridization to these specific regions of the 16S rDNA-genes, as well as to fragments thereof and RNA derived thereof, wherein said oligonucleotides are present in said mixture preferentially in equal amounts.

29. A method for the amplification of 16S rDNA-genes, comprising contacting a sample containing a 16S rDNA-gene with at least one primer, said primer selected from an oligonucleotide consisting of or comprising the sequence SEQ ID NO:1 and/or an oligonucleotide consisting of or comprising the sequence SEQ ID NO:2 as well as their complementary oligonucleotides, reverse oligonucleotides, reverse complementary oligonucleotides, RNA derived thereof, which selectively hybridize to specific regions of the 16S rDNA-genes, as well as to fragments thereof and RNA derived thereof, as well as derivatives thereof, in which 1, 2, 3 or 4 nucleotides are replaced by another nucleotide, added terminally, inserted or deleted without substantially amending the selective hybridization to these specific regions of the 16S rDNA-genes, as well as to fragments thereof and RNA derived thereof, or a mixture of said primers.

30. A method according to claim 29, wherein said sample and a mixture of the primers oligonucleotides SEQ ID NO:1 and SEQ ID NO:2 acting as reverse and forward primer, respectively, are subjected to a polymerase chain reaction (PCR) leading to specific marker fragments with in the range of 100-2000, preferentially in the range of 800-1200 nucleotides.

31. A method according to claim 30, wherein said contacting between the 16S rDNA-gene with the mixture of the pair of oligonucleotides is established under stringent conditions.

32. A method according to claim 31, wherein PCR comprises using recombinant DNA polymerase from Thermus aquaticus, and the stringent conditions comprise 55° C., 500 nM MgCl₂.

33. A method according to claim 30, wherein said contacting comprises as a first step, subjecting the sample containing the 16S rDNA-gene, or the fragments thereof or the RNA derived thereof to heat-denaturation in order to obtain single-stranded chains, bringing these chains into contact with a mixture of the pair of the distinct oligonucleotides, and then extending the chains by means of a DNA polymerase, and wherein the heat-denaturation and the extension cycles are repeated in order to obtain a detectable amount of product.

34. A method for the specific sequencing of 16S rDNA-genes, comprising contacting a sample containing a 16S rDNA-gene with at least one primer, said primer selected from an oligonucleotide consisting of or comprising the sequence SEQ ID NO:1 and/or an oligonucleotide consisting of or comprising the sequence SEQ ID NO:2, as well as their complementary oligonucleotides, reverse oligonucleotides, reverse complementary oligonucleotides, RNA derived thereof, which selectively hybridize to specific regions of the 16S rDNA-genes, as well as to fragments thereof and RNA derived thereof, as well as derivatives thereof, in which 1, 2, 3 or 4 nucleotides are replaced by another nucleotide, added terminally, inserted or deleted without substantially amending the selective hybridization to these specific regions of the 16S rDNA-genes, as well as to fragments thereof and RNA derived thereof, or a mixture of said primers.

35. A method according to claim 34, wherein for sequencing using the chain termination method, the sample is sequenced in both directions using either the reverse, SEQ ID NO:1, or the forward, SEQ ID NO:1, as primer.

36. A method according to claim 35, further comprising amplification of the sample under stringent conditions.

37. A method according to claim 36, wherein the amplification comprises PCR using recombinant DNA polymerase from Thermus aquaticus, and the stringent conditions comprise 52° C., 500 nM MgCl₂.

38. A method for the identification of bacterial species based on their 16S rDNA-gene, said method comprising:
a. isolating a sample material of a bacterial colony and extracting the genetic material or making the genetic material accessible to amplification;
b. subjecting the thus derived material to amplification using at least one primer selected from an oligonucleotide consisting of or comprising the sequence SEQ ID NO:1 and/or an oligonucleotide consisting of or comprising the sequence SEQ ID NO:2 as well as their complementary oligonucleotides, reverse oligonucleotides, reverse complementary oligonucleotides, RNA derived thereof, which selectively hybridize to specific regions of the 16S rDNA-genes, as well as to fragments thereof and RNA derived thereof, as well as derivatives thereof, in which 1, 2, 3 or 4 nucleotides are replaced by another nucleotide, added terminally, inserted or deleted without substantially amending the selective hybridization to these specific regions of the 16S rDNA-genes, as well as to fragments thereof and RNA derived thereof, or a mixture of said primers;
c. purifying the double-stranded DNA amplification products;
d. subjecting the purified product to cycle-sequencing using at least one primer selected from an oligonucleotide consisting of or comprising the sequence SEQ ID NO:1 and/or an oligonucleotide consisting of or comprising the sequence SEQ ID NO:2 as well as their complementary oligonucleotides, reverse oligonucleotides, reverse complementary oligonucleotides, RNA derived thereof, which selectively hybridize to specific regions of the 16S rDNA-genes, as well as to fragments thereof and RNA derived thereof, as well as derivatives thereof, in which 1, 2, 3 or 4 nucleotides are replaced by another nucleotide, added terminally, inserted or deleted without substantially amending the selective hybridization to these specific regions of the 16S rDNA-genes, as well as to fragments thereof and RNA derived thereof, or a mixture of said primers;
e. purifying the sequenced sample; and
f. subjecting the purified and sequenced sample to sequence electrophoresis and signal recording.

39. A method according to claim 38, wherein for the initial amplification in step b) as well as for the sequencing in step d) the same set of primers is used.

40. A method according to claim 38, wherein for the identification of the bacterial genus/species/strains present in the bacterial colony, raw sequence data as obtained in step f) are automatically aligned, noise sequences are optionally stripped, and a consensus sequence is created by comparing the measured data with sequence data from references from a database of known bacterial genus/species/strains.
